(19) United States
(12) Patent Application Publication
(10) Pub. No.: US 2006/0084826 A1
(43) Pub. Date: Apr. 20, 2006

(54) Process for the preparation of naphthylmethylamine derivatives

(75) Inventors: Ramasubramanian Sridharan, Navi Mumbai (IN); Shekhar Bhaskar Bhirud, Navi Mumbai (IN); Vandanapu Loka Appala Purushotham, Navi Mumbai (IN); Ravindra Ashok Janrao, Navi Mumbai (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/215,448

(22) Filed: Aug. 30, 2005

Related U.S. Application Data
(60) Provisional application No. 60/605,588, filed on Aug. 30, 2004.

(51) Int. Cl. C07C 209/06

(52) U.S. Cl. 564/405; 564/406

(57) ABSTRACT

Disclosed is a process for the preparation of a naphthylmethylamine derivative or a pharmaceutically acceptable salt thereof of Formula I 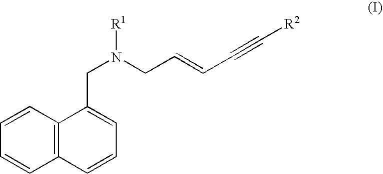
wherein R¹ is a lower straight or branched alkyl group and R² is a lower straight or branched alkyl group, aryl group or araylalkyl group, the process comprising reacting a N-alkyl-1-naphthylmethylamine HCl compound of Formula II: 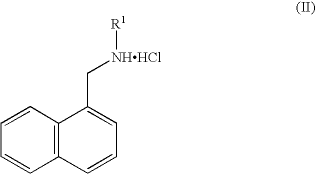
wherein R¹ has the aforestated meaning, with a compound of Formula III 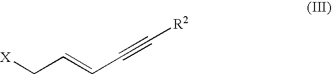
wherein X is a halogen and R² has the aforestated meaning in the presence of at least one base and in at least one solvent.

### PRIORITY

This application claims the benefit under 35 U.S.C. §119 to Provisional Application No. 60/605,588, filed Aug. 30, 2004 and entitled “PROCESS FOR THE PREPARATION OF TERBINAFINE”, the contents of which are incorporated by reference herein.

### BACKGROUND OF THE INVENTION

1. Technical Field

The present invention generally relates to a process for the preparation of naphthylmethylamine derivatives and pharmaceutically acceptable salts thereof.

2. Description of the Related Art

The present invention relates to a process for the preparation of naphthylmethylamine derivatives such as terbinafine (also known as (E)-N-(6,6-dimethyl-2-hepten-4-ynyl)-N-methyl-1-naphthylmethylamine). Terbinafine is represented by the following chemical structure. 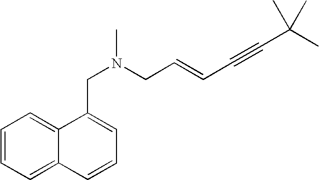
Generally, terbinafine or a pharmaceutical salt thereof is useful as a synthetic allylamine antifungal compound. Terbinafine is indicated for use in the treatment of onychomycosis of toenails and fingernails due to dermatophytes (tinea unguium). Terbinafine hydrochloride is sold under the brand name LAMISIL®.

### SUMMARY OF THE INVENTION

In accordance with one embodiment of the present invention, a process is provided for the preparation of a naphthylmethylamine derivative or a pharmaceutically acceptable salt thereof of Formula I 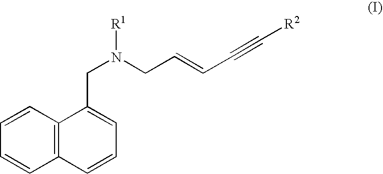
wherein R¹ is a lower straight or branched alkyl group and R² is a lower straight or branched alkyl group, aryl group or araylalkyl group, the process comprising reacting a N-alkyl-1-naphthylmethylamine HCl compound of Formula II: 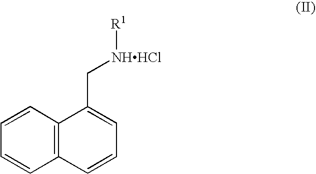
wherein R¹ has the aforestated meaning, with a compound of Formula III 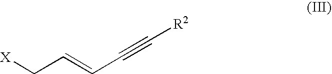
wherein X is a halogen and R² has the aforestated meaning in the presence of at least one base and in at least one solvent.

In accordance with a second embodiment of the present invention, a process for the preparation of terbinafine or a pharmaceutically acceptable salt thereof is provided comprising the reaction of N-methyl-1-naphthalene methanamine HCl with trans-1-halo-6,6-dimethyl-2-heptene-4-yne in the presence of at least one base and in at least one solvent.

### DETAILED DESCRIPTION OF THE INVENTION

One aspect of the present invention provides a process for the preparation of a naphthylmethylamine derivative or a pharmaceutically acceptable salt thereof of Formula I 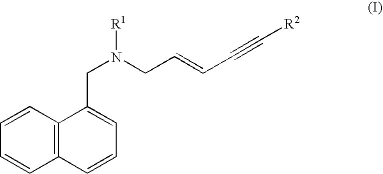
wherein R¹ is a lower straight or branched alkyl group and R² is a lower straight or branched alkyl group, aryl group or araalkyl group. Representative lower alkyl groups include, but are not limited to, straight or branched C₁ to C₈ alkyls such as, for example, methyl, ethyl, n-propyl, isopropyl, n-butyl, isobutyl, sec-butyl, tert-butyl, n-pentyl, isopentyl, neopentyl, tert-pentyl, n-hexyl, and isohexyl, tert-amyl, n-heptyl, n-octyl and the like. Representative aryl groups include, but are not limited to, C₆ to C₁₂ aromatic group such as, for example, phenyl, tolyl, xylyl, biphenyl, naphthyl, and the like. The aryl groups may be substituted with 1 to 3 substituents such as lower alkyls, halogens, hydroxyl groups, alkoxy groups, alkanoyloxy groups, amino groups, cyano groups and the like. Representative aralkyl groups include, but are not limited to, C₁ to C₆ lower alkyls substituted with C₆ to C₁₂ aryl groups as defined above. Examples include benzyl, phenethyl, phenylpropyl and the like, each of which may be optionally substituted.

Generally, the process includes at least reacting a N-alkyl-1-naphthylmethylamine HCl compound of Formula II: 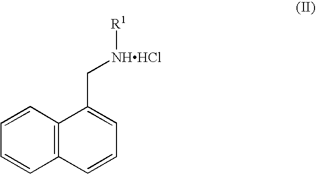
wherein R¹ has the aforestated meaning, with a compound of Formula III: 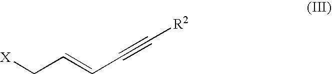
wherein X is a halogen such as, for example, fluorine, chlorine, bromine, iodine and the like, and R² has the aforestated meaning, in the presence of at least one base and in at least one solvent. Generally, the compounds of Formulae II and III are known compounds and can be prepared by any method known in the art and do not constitute a part of the present invention.

A suitable base for use herein can be an inorganic base. Useful inorganic bases include, but are not limited to, alkali or alkaline earth metals carbonates, alkali or alkaline earth metals bicarbonates, hydrides, alkali or alkaline earth metals hydroxides and the like and mixtures thereof. Preferably, the base is sodium hydroxide or potassium carbonate. Generally, the amount of base present in the reaction can range from about 0.5 equivalents to about 6 equivalents and preferably from about 2 equivalents to about 3 equivalents per equivalent of the N-alkyl-1-naphthylmethylamine HCl compound of Formula II.

In one embodiment of the present invention, the solvent can be one or more polyalkylene glycols such as, for example, a polyethylene glycols (PEG) and the like and mixtures thereof. The PEGs can have an average molecular weight ranging from about 200 to about 10,000. In another embodiment of the present invention, the solvent is PEG-400. In yet another embodiment of the present invention, the solvent can be dimethylsulfoxide (DMSO).

The reaction of the compound of Formula II with the compound of Formula III may be carried out at a temperature ranging from about 25° C. to about 100° C., and preferably at a temperature ranging from about 35° C. to about 45° C. The time period for completing the reaction can range from about 3 to about 24. Generally, the compound of Formula II can be reacted with the compound of Formula III in an amount sufficient to form the compound of Formula I, e.g., an amount ranging from about 0.8 equivalents to about 4 equivalents and preferably from about 1.5 equivalents to about 4 equivalents per equivalent of the compound of Formula III.

In a preferred embodiment of the present invention, terbinafine or a pharmaceutically acceptable salt thereof may be prepared by reacting N-methyl-1-naphthalene methanamine HCl of Formula IV: 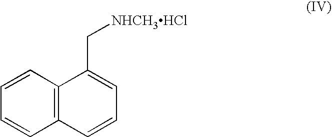
with trans-1-halo-6,6-dimethyl-2-heptene-4-yne of Formula V: 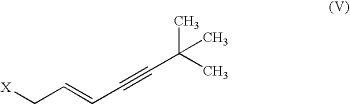
wherein X is a halogen in the presence of at least one base and in at least one solvent as described above.

If desired, the reaction of the compound of Formula II, e.g., N-methyl-1-naphthalene methanamine HCl, with the compound of Formula III, e.g., trans-1-halo-6,6-dimethyl-2-heptene-4-yne, can be carried out in a biphasic medium using a phase transfer catalyst. Generally, phase transfer catalysis (PTC) involves the conversion between chemical species situated in different phases. See, e.g., Dehmlow, E. V.; Dehmlow, S. S., Phase Transfer Catalysis 3^{rd} Ed. (VCH Publishers: NY, 1993). Phase transfer catalysis can carry out reactions between two chemical species in separate phases via a phase transfer catalyst. In one embodiment of the present invention, N-methyl-1-naphthalene methanamine HCl is present in one or more aqueous solvents and trans-1-halo-6,6-dimethyl-2-heptene-4-yne is present in one or more organic solvents. A phase transfer catalyst acts on the trans-1-halo-6,6-dimethyl-2-heptene-4-yne to move it to the organic phase where it reacts with the N-methyl-1-naphthalene methanamine HCl.

Suitable phase transfer catalysts include, but are not limited to, quaternary ammonium salts, quaternary phosphonium salts, polyglycols, crown ethers and the like and mixtures thereof.

As one skilled in the art will readily appreciate, the naphthylmethylamine derivative can then be converted to a pharmaceutically acceptable salt such as a hydrochloride salt, e.g., terbinafine hydrochloride salt, using techniques known in the art.

The following examples are provided to enable one skilled in the art to practice the invention and are merely illustrative of the invention. The examples should not be read as limiting the scope of the invention as defined in the claims.

### EXAMPLE 1

Preparation of Terbinafine

In a 3-necked round bottom flask, N-methyl-naphthylmethylamine hydrochloride (2 g) and PEG-400 (15 ml) were added at room temperature (about 25° C. to about 30° C.) and stirred for about 15 minutes. Trans-1-chloro-6,6-dimethyl-2-heptene-4-yne (2 g) was added to the round bottom flask. A first lot of potassium hydroxide powder (0.2 g) was added to the flask at room temperature and maintained for about 1 hour. A second lot of potassium hydroxide powder (0.4 g) was added to the flask and maintained for about 1 hour. A third lot of potassium hydroxide powder (0.4 g) was added to the flask and the contents of the flask were heated to a temperature ranging from about 35° C. to about 40° C. for about 3 hours. The completion of the reaction was monitored by TLC.

After completion of the reaction as determined by TLC, water (150 ml) was added to the flask. The terbinafine was extracted from the flask with toluene (4 volumes). The toluene layer was washed with 2% tartaric acid solution. The toluene was distilled off under a vacuum and the contents were cooled to room temperature.

### EXAMPLE 2

Preparation of Terbinafine Hydrochloride

Ethyl acetate (4 volumes) was added to the product of Example 1. The solution was cooled to a temperature ranging from about 0° C. to about 5° C. The pH of the reaction mass was adjusted to about 1.5 to about 2 with 5N hydrochloric acid. The reaction mass was maintained for one hour. The reaction mass was then filtered and washed with ethyle acetate (2 volumes). The product was dried at a temperature of about 60° C. Yield=1.5 g.

### EXAMPLE 3

Preparation of Terbinafine

In a round bottom flask equipped with a nitrogen inlet and guard tube, N-methyl-naphthylmethylamine hydrochloride (100 g), dimethylsulfoxide (500 ml) and potassium carbonate (136 g) were added at room temperature (about 25° C. to about 30° C.). Trans-1-chloro-6,6-dimethyl-2-heptene-4-yne (116 g) was added to the round bottom flask over a period of about 15 to about 30 minutes. (Due to the exothermic reaction the temperature rises to about 60° C.). Cool the reaction mass to room temperature and stir for about 4 to about 5 hours. The completion of the reaction was monitored by TLC (RLC mobile phase chloroform:methanol:ammonia at 9:1:one drop of ammonia). If the reaction is not complete, stir for another hour and check again.

After completion of the reaction as determined by TLC, ethyl acetate (400 ml) was added to the flask, followed by an addition of water (3 L). The layers were separated. The aqueous layer was extracted with ethyl acetate (2×300 ml). A 2% tartaric acid solution (400 ml) was added to the combined organic extract wash. The organic layer was washed with water (2×500 ml). Carbon (5 g) was added to the organic layer and stirred at room temperature for about 20 minutes. The reaction mass was filtered with a Celite® bed. The Celite® bed was washed with ethyl acetate (100 ml). The ethyl acetate was distilled out which resulted in about 350 to about 450 ml of reaction mass. The pH of the organic layer was adjusted to about 1.5 to about 2.0 using 5N hydrochloric acid at a temperature ranging from about 0° C. to about 10° C. using a pH meter. The solution was stirred for about 30 minutes.

The compound was filtered and washed with chilled ethyl acetate (100 ml). The crude wet terbinafine was placed in a flask with ethyl acetate (200 ml). The reaction mass was heated to a temperature ranging from about 65° C. to about 75° C. and stirred for about 30 minutes. The compound was filtered and washed with chilled ethyl acetate (50 ml). The compound was slurry-washed with demineralized water (2×300 ml). The compound was dried in an oven at a temperature ranging from about 55° C. to about 60° C. until the moisture content was less than 1%. Yield 80 g.

### EXAMPLE 4

Preparation of Terbinafine Hydrochloride

Acetonitrile (720 ml) was added to the product (80 g) of Example 3 in a round bottom flask equipped with a condenser. The contents were heated to a temperature ranging from about 82° C. to about 87° C. to get a clear solution. The solution was filtered. The reaction mass was cooled to a temperature ranging from about 0° C. to about 5° C. The solution was stirred for about 30 to about 45 minutes. The solution was filtered and washed with chilled acetonitrile (100 ml). The compound was dried at a temperature ranging from about 55° C. to about 60° C. Yield 64 g.

### EXAMPLE 5

Preparation of Terbinafine Hydrochloride

In a round bottom flask, N-methyl-naphthylmethylamine hydrochloride (50 g), toluene (300 ml), water (100 ml), trans-1-chloro-6,6-dimethyl-2-heptene-4-yne (70 g), sodium hydroxide (10.6 g) and tetra butyl ammonium bromide (10 g) were added at room temperature. The reaction mixture was heated to a temperature ranging from about 70° C. to about 80° C. and maintained for about 4 to 5 hours. The completion of the reaction was monitored by TLC (RLC mobile phase chloroform:methanol:ammonia at 9:1:one drop of ammonia). After completion of the reaction as determined by TLC, the aqueous and organic layers were separated. The aqueous layer was extracted with toluene (200 ml). The combined organic layer was washed with water. Toluene from the organic layer was evaporated completely under vacuum and ethyl acetate (150 ml) was added. A 50% hydrochloric acid solution was added to obtain a pH ranging from about 1.0 to about 2.0 at a temperature ranging from about 0° C. to about 5° C. The crystallized hydrochloride salt was filtered and washed with chilled ethyl acetate (100 ml). The compound was dried in an oven at a temperature ranging from about 55° C. to about 60° C. until the moisture content was less than 1%. Yield=25 g.

### EXAMPLE 6

Preparation of Terbinafine Hydrochloride

N-methyl-n-naphthylmethylamine hydrochloride (20 g), dimethyl acetamide (150 ml) and potassium carbonate (K₂CO₃, 45 g) were taken in a dry round bottom flask at room temperature. Trans-1-chloro-6,6-dimethyl-2-heptene-4-yne (20 g) was added to the reaction mixture followed by potassium iodide (KI, 20 g) at a temperature of about 25° C. The reaction mass was stirred for 24 hours. The completion of the reaction was monitored by TLC (RLC mobile phase chloroform:methanol:ammonia at 9:1:one drop of ammonia). After completion of the reaction as determined by TLC, water (1500 ml) was added and the product was extracted with ethyl acetate (4×20 ml). After the layer separation the combined organic layer was washed with 2% tartaric acid solution followed by water. The layer was cooled to a temperature ranging from about 0° C. to about 5° C. The pH was adjusted to a range between about 1.5 and 2.0 with a 5N HCL solution. The solution was stirred for a period of about 1 hour at a temperature ranging from about 0° C. to about 5° C. The solution was filtered and washed with chilled ethyl acetate (40 ml) and dried in an oven at a temperature ranging from about 55° C. to about 60° C. until the moisture content was less than 1%. Yield=15 g.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. A process for the preparation of a naphthylmethylamine derivative or a pharmaceutically acceptable salt thereof of Formula I 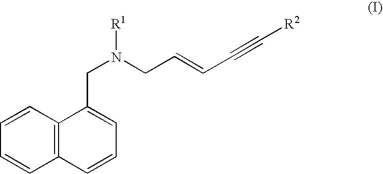
wherein R¹ is a lower straight or branched alkyl group and R² is a lower straight or branched alkyl group, aryl group or araylalkyl group, the process comprising reacting a N-alkyl-1-naphthylmethylamine HCl compound of Formula II: 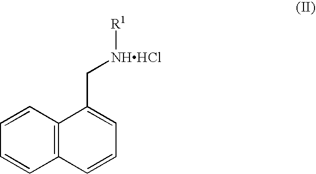
wherein R¹ has the aforestated meaning, with a compound of Formula III 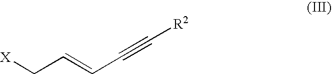
wherein X is a halogen and R² has the aforestated meaning in the presence of at least one base and in at least one solvent.

2. The process of claim 1, wherein R¹ is a straight or branched C₁ to C₈ alkyl group.

3. The process of claim 1, wherein R¹ is a straight or branched C₁ to C₈ alkyl group and R² is a straight or branched C₁ to C₈ alkyl group.

4. The process of claim 1, wherein the N-alkyl-1-naphthylmethylamine HCl compound of Formula II is N-methyl-1-naphthalene methanamine HCl of Formula IV: 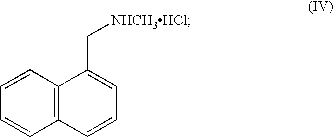
and the compound of Formula III is trans-1-halo-6,6-dimethyl-2-heptene-4-yne of Formula V: 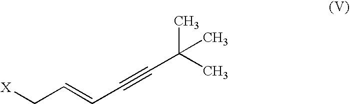
wherein X is selected from the group consisting of fluorine, chlorine, bromine and iodine.

5. The process of claim 1, wherein the base is an inorganic base.

6. The process of claim 5, wherein the inorganic base is selected from the group consisting of an alkali metal carbonate, alkaline earth metal carbonate, alkali metal bicarbonate, alkaline earth metal bicarbonate, alkali metal hydride, alkali metal hydroxide, alkaline earth metal hydroxide and mixtures thereof.

7. The process of claim 1, wherein the base is sodium hydroxide or potassium carbonate.

8. The process of claim 1, wherein the solvent is a polyalkylene glycol.

9. The process of claim 8, wherein the polyalkylene glycol is a polyethylene glycol.

10. The process of claim 1, wherein the solvent is PEG-400.

11. The process of claim 1, wherein the solvent is dimethylsulfoxide

12. The process of claim 1, wherein the temperature of the reaction is maintained at about 25° C. to about 100° C.

13. The process of claim 1, wherein the reaction is carried out in the presence of a phase transfer catalyst.

14. The process of claim 13, wherein the phase transfer catalyst is selected from the group consisting of a quaternary ammonium salt, quaternary phosphonium salt, polyglycol, crown ether and mixtures thereof.

15. The process of claim 1, wherein the naphthylmethylamine derivative of Formula I is thereafter converted to a pharmaceutically acceptable salt thereof.

16. The process of claim 1, wherein the naphthylmethylamine derivative of Formula I is thereafter converted to a hydrochloride salt.

17. The process of claim 1, wherein the naphthylmethylamine derivative of Formula I is terbinafine.

18. The process of claim 17, wherein terbinafine is thereafter converted to a pharmaceutically acceptable salt thereof.

19. The process of claim 17, wherein terbinafine is thereafter converted to a hydrochloride salt.

20. A process for the preparation of terbinafine comprising reacting N-methyl-1-naphthalene methanamine HCl of Formula IV: 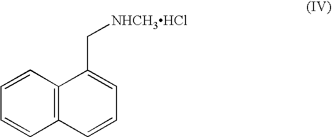
with trans-1-halo-6,6-dimethyl-2-heptene-4-yne of Formula V: 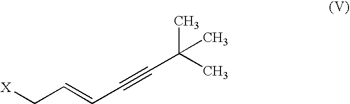
wherein X is a halogen in the presence of at least one base and in at least one solvent.

21. The process of claim 20, wherein the base is selected from the group consisting of an alkali metal carbonate, alkaline earth metal carbonate, alkali metal bicarbonate, alkaline earth metal bicarbonate, alkali metal hydride, alkali metal hydroxide, alkaline earth metal hydroxide and mixtures thereof.

22. The process of claim 20, wherein the base is sodium hydroxide or potassium carbonate.

23. The process of claim 20, wherein the solvent is a polyethylene glycol.

24. The process of claim 20, wherein the solvent is a polyethylene glycol.

25. The process of claim 20, wherein the solvent is PEG-400.

26. The process of claim 20, wherein the solvent is dimethylsulfoxide.

27. The process of claim 20, wherein the temperature of the reaction is maintained at about 25° C. to about 100° C.

28. The process of claim 20, wherein terbinafine is thereafter converted to a pharmaceutically acceptable salt thereof.

29. The process of claim 20, wherein terbinafine is thereafter converted to a hydrochloride salt.
